(19) United States
(12) Patent Application Publication
(10) Pub. No.: US 2008/0131500 A1
(43) Pub. Date: Jun. 5, 2008

(54) METHODS AND COMPOSITIONS FOR RAPID INACTIVATION OF PROTEINS

(75) Inventor: ROWEN J.-Y. CHANG, Houston, TX (US)

Correspondence Address: FULBRIGHT & JAWORSKI L.L.P., 600 CONGRESS AVE., SUITE 2400, AUSTIN, TX, 78701, US

(73) Assignee: The Board of Regents of The University of Texas System

(21) Appl. No.: 11/566,595

(22) Filed: Dec. 4, 2006

(51) Int. Cl. A61K 9/48; A61P 25/28; A01P 1/00; A61K 47/02; A61K 39/395; C07K 14/00; A01N 25/00

(52) U.S. Cl. 424/451; 530/410; 530/408; 530/402; 530/409; 514/769; 424/130.1; 424/45

(57) ABSTRACT

Disclosed are methods of inactivating a protein, such as cleaving a disulfide bond of a protein, that involve contacting the protein with a reducing agent, a denaturant, and a hydroxide ion, wherein the pH of the composition is about 10.0 to about 14.0. Also disclosed are methods of treating or preventing a disease in a subject, such as a toxin-related disease or a prion-related disease, that involve contacting a subject with a pharmaceutically effective amount of a reducing agent, a denaturant, and a hydroxide ion, wherein the pH of the composition is about 9.0 to about 14.0. Also disclosed are compositions that include a reducing agent, a denaturant, and a hydroxide ion, wherein the pH of the composition is about 9.0 to about 14.0.

### BACKGROUND OF THE INVENTION

1. Field of the Invention

The present invention relates generally to the fields of protein chemistry, toxins, and prions. More particularly, it concerns compositions and methods of inactivating a protein or cleaving a disulfide bond of a protein that involve a reducing agent, a denaturant, and a hydroxide ion at a pH of 10.0 to 14.0. It also concerns methods of treating or preventing a disease in a subject that involve contacting the subject with a pharmaceutically effective amount of a composition that includes a reducing agent, a denaturant, and a hydroxide ion.

2. Description of Related Art

Many naturally occurring toxins and poisonous compounds are proteins that contain disulfide bonds. These compounds include a wide range toxic proteins isolated from plants, bacteria, venom of reptiles and insects, etc. Some of these toxic proteins have found their benefit in disease treatment, but many of them are harmful or lethal upon inadvertent contact and injection to human. Specifically some plant toxins such as ricin (Audi et al., 2005; Mantis, 2005) from castor beans and related abrin, have caused serious concerns as potential weapons of terror attack and biological warfare due to the simple process of their production and their worldwide availability (Bigalke and Rummel, 2005). Consequently, development of an efficient and effective method of inactivation of these toxic proteins is imperative.

Active disulfide proteins can be inactivated by different methods. For example, they can be inactivated by denaturation without covalent modification of the protein. This is accomplished by various approaches, including application of chemical denaturants, heat, extreme pH, detergents or organic solvents (Pace et al., 1989; Volkin and Klibanov, 1989). However, most processes of denaturation are readily reversible. For instance, proteins denatured by chemical denaturants (urea or guanidinium chloride) or heat usually renature and refold spontaneously following removal of the denaturant and decrease of the temperature.

Alternatively, active disulfide proteins can be inactivated by proteolysis. The process is irreversible, but it typically takes hours and requires enzymes that have to be dealt with latter. Disulfide proteins can be inactivated by chemical reduction of their disulfide bonds. However, this process is also reversible because fully reduced proteins with intact cysteines are usually capable of renaturation via oxidative folding (White, 1972; Creighton, 1986), if appropriate conditions are given.

Thus, most disulfide-containing proteins can be permanently inactivated only if their disulfide bonds are covalently modified or annihilated. Although such methods are available, they require either highly noxious chemicals or work in a relatively sluggish fashion. For example, the cleavage of disulfide bonds by performic acid oxidation requires highly acidic solution (Spackman et al., 1960). The method of reduction and alkylation entails tedious and stepwise chemical reactions (Waxdal et al., 1968).

One example of a disulfide-containing protein is a prion. A “prion” is defined herein to refer to a proteinaceous-infectious agent that causes relatively similar brain diseases in humans and/or in animals, which are invariably fatal. These diseases include Creutzfeldt-Jakob disease (CJD) and variant CJD (vCJD) in humans, bovine spongiform encephalopathy (BSE) in cattle, also know as “Mad Cow Disease,” scrapie in sheep, and wasting disease in elk. These diseases affect the neurological system of the animal or animals and are characterized by initially long incubation times followed by a short period of neurological symptoms, including dementia and loss of coordination, and eventually death.

The infectious agent responsible for these diseases is thought to be a simple protein, with no associated nucleic acids. The pathogenic mechanism for prion diseases has been proposed to involve an initially normal host encoded protein that undergoes a conformational change to become an abnormal form (a prion), which has the ability of self-propagation. The abnormal form of the protein is not broken down effectively in the body and its accumulation in certain tissues (in particular neural tissue) eventually causes tissue damage and the associated clinical signs.

There are currently no known effective treatments for prion diseases in animals or humans, and death thus follows the onset of neurological symptoms. Progress in the identification of target treatment drugs has been slow, due to the inability to perform testing in vitro. In addition, because these diseases tend to be animal specific, it is not known whether tests done on animals can be readily applied to humans. There have been a few reports of methods for decontamination of surfaces contaminated with prion-infected material (see, e.g., U.S. Pat. No. 7,071,152 and U.S. Pat. No. 7,001,873). These methods are limited by requiring prolonged contact of the active agent with the surface or numerous steps.

Prions are notoriously very hardy and demonstrate resistance to routine methods of decontamination and sterilization. There is a clear need for products and processes that are effective against prions.

Therefore, there is the need for more efficient and effective methods of inactivating disulfide containing proteins. Such methods can be applied in the rapid inactivation of toxins and poisonous proteins that pose a threat to humans

### SUMMARY OF THE INVENTION

The inventor has identified novel compositions and methods that can be applied in the rapid inactivation of proteins. In particular, a novel composition has been identified which enables a rapid (e.g., 20-30 seconds), irreversible, and quantitative inactivation of disulfide containing proteins. The reaction can take place at room temperature (20° C. to 25° C.). The formula includes a denaturant, a reductant and hydroxide ion. The component of hydroxide ion serves two major functions. First, it accelerates the cleavage of disulfide bonds mediated by the reducing agent and denaturant, leading to an instant inactivation of disulfide proteins. Second, it triggers a rapid covalent destruction of sulfhydryl groups and disulfide bonds via the mechanism of base catalyzed β-elimination, thus leading to the permanent inactivation of toxic disulfide proteins. Usefulness of this invention has been demonstrated with the effective and rapid inactivation of numerous highly stable disulfide containing proteins, including cardiotoxin, as discussed in the Examples below.

The present invention generally pertains to methods for inactivating a molecule, such as a protein, that involves contacting the molecule with a composition that includes a reducing agent, a denaturant, and a hydroxide ion, wherein the pH of the composition is about 10.0 to about 14.0 and wherein contacting results in inactivation of the protein.

The present invention is also generally directed to methods for cleaving a disulfide bond of a protein, that involves contacting the protein with a composition that includes a reducing agent, a denaturant, and a hydroxide ion, wherein the pH of the composition is about 10.0 to about 14.0 and wherein contacting results in inactivation of the protein.

The phrase “inactivating a protein” refers to reduction or loss of at least some of the original properties of the protein, such as its biological activity. In particular, inactivation of a protein may be the result of cleavage of one or more disulfide bonds (also called SS-bonds or disulfide bridges) of the protein. A disulfide bond is a covalent bond between sulfur atoms that binds two polypeptides or different parts of one polypeptide and is a structural determinant in many protein molecules. Disulfide bonds in proteins are formed, for example, between the thiol groups of cysteine residues. The prototype of a protein disulfide bond is the two-amino-acid peptide, cystine, which is composed of two cysteine amino acids joined by a disulfide bond. The inactivation of the protein may be reversible or irreversible. In particular embodiments, the compositions of the present invention result in irreversible inactivation of a protein

The methods set forth herein can be applied in any situation where inactivating a protein is desired. Non-limiting examples of such situations include treatment of items, such as medical equipment, pharmaceutical equipment, mortuary equipment, meat processing equipment, food handling equipment, and the like that are known or suspected to be contaminated with a protein for which inactivation is desired. In some embodiments, the methods are directed to decontamination of a surface that is known or suspected to be contamination with a toxic protein, wherein contamination is the result of a bioterrorist attack. The surface may be an organic surface or a non-organic surface. The methods set forth herein are also suitable for treatment of medical instruments and devices which have been employed in a surgical operation, such as scalpels, scissors, endoscopes, forceps, catheters, retractors, clamps, spatulas, and so forth.

A “reducing agent” is defined herein to refer to any molecule that reduces another molecule by donating one or more electrons. A reducing agent acts as an electron donor in an oxidation-reduction reaction. The reducing agent can be any reducing agent known to those of ordinary skill in the art. In some embodiments, the reducing agent is a thiol, a phosphine, or a phosphite. Examples of thiols include dithiothreitol (DTT), 5,5′-dithiobis-(2-nitrobenzoic acid), ethanedithiol, 2-mercaptoethanol, 2-mercaptoethylamine, and thioglycolic acid. Examples of phosphines include tris-carboxyethylphosphine, trimethyl phosphine, triethyl phosphine, triphenyl phosphine, and tributylphosphine. An example of a phosphite is triethyl phosphite.

Any concentration of reducing agent is contemplated in the compositions of the present invention. For example, the concentration of the reducing agent may be at least about 0.01 mM, 0.02 mM, 0.04 mM, 0.06 mM, 0.08 mM, 0.10 mM, 0.12 mM, 0.14 mM, 0.16 mM, 0.20 mM, 0.25 mM, 0.30 mM, 0.35 mM, 0.40 mM, 0.45 mM, 0.50 mM, 0.55 mM, 0.60 mM, 0.65 mM, 0.70 mM, 0.75 mM, 0.80 mM, 0.85 mM, 0.90 mM, 0.95 mM, 1.00 mM, 0.002 M, 0.004 M, 0.006 M, 0.008 M, 0.01 M, 0.02 M, 0.04 M, 0.06 M, 0.08 M, 0.10 M, 0.20 M, 0.40 M, 0.60 M, 0.80 M, 1.0 M, 1.2 M, 1.4 M, 1.6 M, 1.8 M, 2.0 M, 2.2 M, 2.4 M, 2.6 M, 2.8 M, 3.0 M, 3.2 M, 3.4 M, 3.6 M, 3.8 M. 4.0 M, 4.2 M, 4.4 M, 4.8 M, 5.0 M, 5.2 M, 5.4 M, 5.6 M, 5.8 M, 6.0 M, 6.2 M, 6.4 M, 6.6 M, 6.8 M, 7.0 M, or greater, or at least any intervening concentration between any two recited concentrations, or a concentration within any range of concentrations derivable herein. For example, the concentration of reducing agent may be about 0.1 mM to about 5.0 M. In some embodiments, the concentration of reducing agent is about 1 mM to about 1M.

A “denaturant” is defined herein to refer to an agent that causes the tertiary structure of a protein to unfold. This may or may not result in loss of at least some of the original properties of the protein, such as its biological activity. The denaturant can be any compound known to those of ordinary skill in the art. In particular embodiments, the denaturant is urea, thiourea, guanidinium chloride, imidazole, formamide, dimethylsulfoxide, or a thiocyanate. An example of a thiocyanate is guanidine thiocyanate.

Any concentration of denaturant is contemplated in the compositions of the present invention. For example, the concentration of the denaturant may be at least 0.01 mM, 0.02 mM, 0.04 mM, 0.06 mM, 0.08 mM, 0.10 mM, 0.12 mM, 0.14 mM, 0.16 mM, 0.20 mM, 0.25 mM, 0.30 mM, 0.35 mM, 0.40 mM, 0.45 mM, 0.50 mM, 0.55 mM, 0.60 mM, 0.65 mM, 0.70 mM, 0.75 mM, 0.80 mM, 0.85 mM, 0.90 mM, 0.95 mM, 1.00 mM, 0.002 M, 0.004 M, 0.006 M, 0.008 M, 0.01 M, 0.02 M, 0.04 M, 0.06 M, 0.08 M, 0.10 M, 0.20 M, 0.40 M, 0.60 M, 0.80 M, 1.0 M, 1.2 M, 1.4 M, 1.6 M, 1.8 M, 2.0 M, 2.2 M, 2.4 M, 2.6 M, 2.8 M, 3.0 M, 3.2 M, 3.4 M, 3.6 M, 3.8 M. 4.0 M, 4.2 M, 4.4 M, 4.8 M, 5.0 M, 5.2 M, 5.4 M, 5.6 M, 5.8 M, 6.0 M, 6.2 M, 6.4 M, 6.6 M, 6.8 M, 7.0 M, or greater, or at least any intervening concentration between any two recited concentrations, or a concentration within any range of concentrations derivable herein. For example, the concentration of denaturant may be about 0.1 mM to about 5.0 M. In some embodiments, the concentration of denaturant is about 1 mM to about 1.0 M.

The compositions of the present invention also include a hydroxide ion. For example, the hydroxide ion may be incorporated as NaOH or KOH. The concentration of hydroxide ion may be at least about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 13.4, 12.5, 12.6, 12.7, 12.8, 12.9, 13.0, 13.1, 13.2, 13.3, 13.4, 13.5, 13.6, 13.7, 13.8, 13.9, 14.0, or at least any pH derivable between any of the aforementioned pH's, or any range of pH derivable therein. In certain embodiments, the pH of the composition is about 10.0 to about 14.0. In other embodiments, the pH of the composition is about 11.0 to about 14.0. In further embodiments, the pH of the composition is about 12.0 to about 14.0 In still further embodiments, the pH of the composition is about 13.0 to about 14.0. In particular embodiments, the concentration of hydroxide ion is about 0.1N to about 10N. In more particular embodiments, the concentration of hydroxide ion is about 0.5N to about 1.5N.

Some embodiments of the compositions of the present invention include DTT and NaOH. In some more specific embodiments, the composition further includes guanidinium chloride. Any concentration of guanidinium chloride may be present in these embodiments. For example, the concentration of guanidinium chloride in the composition may be about 0.1M to about 8M. In further embodiments, the concentration of guanidinium chloride in the concentration is about 0.2M to about 6.0 M.

Further embodiments of the compositions of the present invention include DTT, NaOH, and guanidine thiocyanate. Any concentration of guanidine thiocyanate may be present in these embodiments. For example, the concentration may be about 0.01M to about 6 M. In further embodiments, the concentration of guanidine thiocyanate is about 0.1 M to about 1M. The concentration of DTT in these embodiments may be about 0.1 mM to about 5M. In some embodiments, the concentration of DTT is about 1mM to about 1M.

In particular embodiments of the present invention, the composition is further defined as a disinfectant. A “disinfectant” is an agent or composition that removes, kills, destroys, or otherwise render non-pathogenic a pathogen. As used herein, and unless stated otherwise, the term “pathogen” includes viruses, bacteria, fungi, prions, prion related proteins, and other micro-organisms capable of exerting pathogenic effects in multi-cellular organisms. Thus, the use of the term “pathogen” contemplates micro-organisms capable of causing disease in mammals, including humans.

The composition may be formulated in any manner known to those of ordinary skill in the art. Non-limiting examples include a liquid, a gel, a foam, a spray, a mist, or a vapor. For example, the composition may be sprayed on a surface that is known or suspected of being contaminated with a protein, painted onto the surface with a brush or other applicator, or an object known or suspected to be contaminated with a protein may be dipped or immersed into the composition. The spray, for example, may be a liquid spray, a mist, a vapor, or an aerosol. In other embodiments, the composition is poured into a liquid that includes or is suspected of including a protein to be inactivated. Thus, for example, the compositions set forth herein can be applied in the inactivation of a protein on a surface, in the air, or in a liquid.

The composition may include one or more additional agents. Non-limiting examples of these additional agents include a thickener, a corrosion inhibitor, a chelating agent, a polymer, a humectant, a surfactant, or an antimicrobial agent. Non-limiting examples of thickeners include cellulose derivatives, acrylic acid-based polymers, gums, such as guars, guar derivatives, alginates, alginate derivatives, non-ionic surfactants, non-ionic polymers, and combinations thereof. Non-limiting examples of humectants include sorbitol, glycerine, glucitol, polyethylene glycol, propylene glycol, dipropylene glycose, 1,3-butylene glycol, hexylene glycol, and mixtures thereof. The polymer, for example, may be a cationic polymer, such as carboxylated polymers, vinyl addition polymers, or dialkyldiallyl ammonium salt homopolymers. Non-limiting examples of chelating agents include carboxylic acid-based polymers, such as polyacrylic acid, ethylenediaminetetraacetic acid (EDTA), hexametaphosphate, or salts thereof. Non-limiting examples of surfactants include anionic, cationic nonionic and zwitterionic surfactants. Non-limiting examples of metal corrosion inhibitors are silicic acid salts and phosphoric acid salts. Exemplary antimicrobial agents include a phenol, quaternary ammonium compound, or an oxidizing agent, such as sodium hypochlorite, hydrogen peroxide, or peracetic acid.

The composition may be aqueous or nonaqueous. In some embodiments, the compositions include a mixture of aqueous and organic solvents. In some embodiments, the composition is further defined as a cleaning composition, suitable for removing any fixed proteinaceous matter such as clumps of protein from a surface. Thus, in some embodiments, the composition both deactivates a protein and removes the protein from a surface.

The protein to be inactivated can be any protein known to those of ordinary skill in the art. In particular embodiments of the present invention, the protein includes one or more disulfide linkages. For example, in some embodiments, the protein includes 1-20 disulfide bonds. In more particular embodiments, the protein includes 5-10 disulfide bonds.

For example, the protein may be a prion, and the method of inactivating a protein is further defined as a method for inactivating a prion. Inactivation of a prion may involve depolymerizing aggregates of the prion protein. In further embodiments, the protein is a toxin, and the method is further defined as a method for inactivating a toxin. A “toxin” or “toxic substance” or “poison” or “poisonous substance” is defined herein to refer to any compound that can cause disease or death of a subject. The toxin can be any substance known to those of ordinary skill in the art. For example, the toxin may be ricin, abrin, botulinum toxin, cholera toxin, snake venom toxin, cardiotoxin, diphtheria toxin, Bacillus larval toxin, yeast killer toxin, K1 killer toxin, Cerebratulus toxin, pertussis toxin, hemolysin toxin, microbial-mucosal toxin, Shiga toxin, Helicobacter pylori vacA toxin, anthrax toxin, or tetanus toxin.

In some embodiments of the present invention, the methods for inactivating a protein is further defined as a method of treating or preventing a disease or health-related condition in a subject, wherein the protein is in the subject or on a surface of the subject, involving administering to the subject a pharmaceutically effective amount of the composition. The subject may be any subject, such as a vertebrate. The vertebrate may be a mammal. In particular embodiments, the mammal is a human. For example, the disease or health-related condition to be treated or prevented may be the signs and symptoms associated with exposure to a toxin.

Any method or route of administration known to those of ordinary skill in the art is contemplated by the present invention. For example, administering may involve topical, aerosol, local, intravenous, intracardiac, intradermal, intralesional, intrathecal, intracranial, intrapericardial, intraumbilical, intraocular, intraarterial, intraperitoneal, intratumor, subcutaneous, intramuscular, or intravitreous administration. In particular embodiments, a pharmaceutically effective amount of the composition is topically applied to a surface of the subject. For example, the composition may be sprayed on a surface of the subject.

The composition may be formulated in any manner known to those of ordinary skill in the art. For example, the composition may be formulated as a liquid, a gel, an an ointment, a cream, a shampoo, a rinse, a spray, a mist, or a vapor. In particular embodiments, the composition is formulated as a nanocapsule. A “nanocapsule” as used herein refers to a small container, generally about 1 nm to about 100 μm in size, that encloses a particular amount of a pharmaceutical agent or pharmaceutical composition. Nanocapsules can generally entrap compounds in a stable and/or reproducible way. In some embodiments, the nanocapsule includes a tissue-targeting ligand attached to the outer surface of the nanocapsule. Any tissue-targeting ligand known to those of ordinary skill in the art is contemplated by the present invention. In particular embodiments, the targeting ligand is an antibody. For example, the antibody may be directed against the protein to be inactivated or may be directed to a particular tissue in a subject. To avoid side effects due to intracellular polymeric overloading, such ultrafine particles should be designed using polymers able to be degraded in vivo. For example, the nanocapsule can include polyalkyl-cyanoacrylates. Nanocapsules are discussed at length elsewhere in this specification.

In certain embodiments of the present invention, the method for inactivating a protein or cleaving a disulfide bond of a protein is further defined as a method for treating or preventing a prion-related disease in a subject, wherein contacting the prion with the compound results in treatment or prevention of a prion-related disease in the subject. The prion-related disease can be any prion-related disease known to those of ordinary skill in the art. For example, the prion-related disease may be Creutzfeldt-Jakob Disease, bovine spongiform encephalopathy, Gerstmann-Straussler-Scheinker Syndrome, kuru, scrapie, fatal familial insomnia, or a variant of any of these diseases.

In some embodiments, the method of inactivating a protein or cleaving a disulfide bond of a protein is further defined as a method for disinfecting a surface that has been exposed to an infectious agent, wherein the infectious agent includes a protein that includes a disulfide bond and wherein the composition is sprayed onto the surface. In further embodiments, the method is further defined as a method for detoxifying a surface that has been exposed to a toxin, wherein the toxin includes a disulfide bond and wherein the composition is sprayed onto the surface. The surface may be an organic surface, or a non-organic surface. Examples of organic surfaces include skin or mucosal surfaces of a subject. In still further embodiments, the method is further defined as a method for disinfecting or detoxifying a liquid, wherein the composition is added to the liquid. In particular embodiments, the method of inactivating a protein or method of cleaving a disulfide bond of a protein is further defined as a method for counteracting a bioterrorist attack, wherein the compound is a biological weapon.

The present invention is also generally directed to compositions that include a reducing agent, a denaturant, and a hydroxide ion, wherein the pH of the composition is about 10.0 to about 14.0. The denaturant and reducing agent can be any of the agents set forth above. In particular embodiments, the concentration of the reducing agent is 100 mM or less. In further embodiments, the concentration of the denaturant is about 2.0 M or less. As set forth above, the composition may be a liquid, a gel, a foam, a spray, a mist, or any other formulation known to those of ordinary skill in the art.

In certain embodiments, the pH of the composition is about 10.0 to about 14.0, or any of those concentrations set forth above. In specific embodiments, the pH of the composition is about 11.0 to about 14.0. In more specific embodiments, the pH of the composition is about 12.0 to about 14.0. In even more specific embodiments, the concentration of hydroxide ion is about 0.1N to about 10N.

The composition of the present invention may be contained in a suitable container means. For example, in some embodiments, the container is a container, such as a cannister, suitable for spraying the composition. For example, the container may be configured for spraying the composition as a liquid spray or aerosol.

The present invention is also generally directed to kits include one or more containers containing a particular amount of one of the compositions of the present invention. In some embodiments, the kit includes a first container means including a composition that includes a reducing agent, a second container means including a composition that includes a denaturant, and a third container means including a composition that includes hydroxide ion. The compositions are combined at the time of use. The kit may or may not include a means for applying the composition, such as a spray nozzle or other applicator. In further embodiments, the kit includes nanocapsules as set forth above in a suitable container means. The kit may further include packaging for shipping the container, and instructions regarding methods for use of the composition.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1. The underlying mechanism of rapid and irreversible inactivation of toxic disulfide proteins. Step (1) The concerted actions of denaturant, reductant and hydroxide ion prompt a rapid inactivation by N to R conversion. Step (2) The hydroxide ion triggers an irreversible destruction of sulfhydryl groups of R-protein via the mechanism of base catalyzed β-elimination, leading to the formation of dehydroalanine. Step (3) Coupling of dehydroalanine with the side chains of lysine and cysteine forms permanently inactivated XX, which comprises denatured monomers, dimers and trimers that are intra- and inter-crosslinked by lysinoalanine and lanthionine.

FIG. 2. Demonstration that increased pH accelerates the reduction of disulfide bonds and triggers the covalent destruction of sulfhydryl groups. N-Hirudin (1 mg/ml) was reduced with 50 mM DTT at 22° C. in solutions of pH 9.0, pH 11.0 and pH 13.5. The buffers of pH 9.0 and 11.0 were prepared by the titration of 0.1N NaHC0₃ and 0.1N NaOH. The samples were quenched at different time points by acidification and analyzed by HPLC using the following conditions. Solvent A for HPLC was water containing 0.1% trifluoroacetic acid. Solvent B was acetonitrile/water (9:1, by volume) containing 0.086% trifluoroacetic acid. The gradient was 27% to 36% solvent B linear in 25 min. The flow rate was 0.5 ml/min. The column was Zorbax 300SB C-18 for peptides and proteins, 4.6 mm, 5 pm. Column temperature was 22° C. N indicates native hirudin. R indicates fully reduced hirudin. XX indicates complex structures of hirudin resulted from base catalyzed β-elimination of R-hirudin.

FIG. 3. Demonstration that inclusion of denaturant further accelerate the reduction and irreversible destruction of disulfide bonds. N-Hirudin (1 mg/ml) was incubated at 22° C. in a solution containing 1N NaOH (pH 13.5), 50 mM DTT and different concentrations of guanidine thiocyanate (GdnSCN). The samples were quenched at various time points by acidification and analyzed by HPLC. N indicates native hirudin. R indicates fully reduced hirudin. XX indicates complex structures of hirudin resulted from base catalyzed β-elimination of R-hirudin.

FIG. 4. Inactivation of N-hirudin by the optimized composition of denaturant, reductant and hydroxide ion. (Right panel) N-Hirudin (1 mg/ml) was incubated at 22° C. in a solution containing 1N NaOH (pH 13.5), 100 mM DTT and 1M GdnSCN. (Left panel) N-Hirudin (1 mg/ml) was incubated at 22° C. in a solution containing 0.5N NaOH (pH 13.5), 50 mM DTT and 0.5M GdnSCN. The samples were quenched at various time points by acidification and analyzed by HPLC. N indicates native hirudin. R indicates fully reduced hirudin. XX indicates complex structures of hirudin resulted from base catalyzed β-elimination of R-hirudin.

FIG. 5. Demonstration of the chemical reversibility of inactivated hirudin and CTX-III. N-Hirudin and N-CTX-III (cardiotoxin III; 1 mg/ml) were incubated at 22° C. for 1 min, 5 min, 15 min and 60 min, in a solution containing 1N NaOH (pH 13.5), 100 mM DTT and 1M GdnSCN. The inactivated samples were quenched by acidification. They were separated from the denaturant, reductant and hydroxide ion by gel filtration eluted with 0.5% aqueous trifluoroacetic acid. The samples were freeze-dried and then reconstituted (0.5 mg/ml) in Tris-HCl buffer (0.1M, pH 8.4) containing GSH/GSSG (1 mM/0.5 mM) to allow oxidative folding at 22° C. overnight. The refolded samples were again quenched by acidification and analyzed by HPLC. N indicates native hirudin and native CTX-III respectively.

FIG. 6. Inactivation of Cardiotoxin-III (CTX-III) by the optimized composition of denaturant, reductant and hydroxide ion. Native CTX-III (1 mg/ml) was incubated at 22° C. in a solution containing 1N NaOH (pH 13.5), 100 mM DTT and 1M GdnSCN. The samples were quenched at various time points by acidification and analyzed by HPLC. N indicates native CTX-III. R indicates inactivated and fully reduced CTX-III. XX indicates complex structures of CTX-III resulted from base catalyzed β-elimination of R-CTX-III.

FIG. 7. Inactivation of bovine pancreatic trypsin inhibitor (BPTI), human epidermal growth factor (EGF), bovine ribonuclease A (RNase A) and leech secretory leucocyte protease inhibitor (SLPI). Native proteins (1 mg/ml) were incubated at 22° C. in a solution containing 1N NaOH (pH 13.5), 100 mM DTT and 1M GdnSCN. The samples were quenched at various time points by acidification and analyzed by HPLC. N indicates native proteins. R indicates inactivated and fully reduced proteins. XX indicates complex structures of inactivated proteins resulted from base catalyzed β-elimination of R-proteins.

FIG. 8A, 8B, 8C. De-Aggregation of a polymerized form of recombinant mouse prion protein (mPrP-Z) to form mPrP-M. FIG. 8A—In a solution containing 1N NaOH (pH 13.5), 100 mM DTT and 1M GdnSCN. FIG. 8B—In a solution containing 0.1M Tris-HCl (pH 8.0), 100 mM DTT and 6M GdnCI. FIG. 8C—In a solution containing 0.1M Tris-HCl (pH 8.0) and 6M GdnCI. Purified mPrP-z was freeze-dried and dissolved in the above mentioned solutions. The prion protein concentration was 1 mg/ml and the reactions were carried out at 22° C. The reactions were trapped at different time points by mixing aliquots of the sample with 8 volumes of 4% aqueous trifluoroacetic acid, and analyzed by size-exclusion chromatography (TSK-Gel, G3000SWXL, 7.8 mm×30 cm, 5 μm), eluted with Acetonitrile/0.1% aqueous TFA (40:60, by volume). “Z” denotes MPrP-Z. “M” indicates monomeric form of mPrP in reduced form.

### DESCRIPTION OF ILLUSTRATIVE EMBODIMENTS

About 70% of all extra-cellular proteins contain disulfide bonds. Most toxic proteins isolated from plants, reptiles and insects are also disulfide containing proteins. The present invention is based on the inventor's identification of compositions that can be applied in the rapid and irreversible inactivation of disulfide-containing proteins. The inactivation can take place at room temperature. A composition for rapid and irreversible inactivation of a disulfide-containing molecule as described in this invention should have a wide range of potential applications. For example, the composition can be applied in counter terror attacks that involve toxic or poisonous proteins that contain one or more disulfide bonds. For instance, a liquid spray of this invention can be used to inactivate toxic proteins such as ricin, abrin or botulinum that are known as biological weapons. The composition can also be applied in disease treatment and prevention. For instance, the composite can be packaged in a nanocapsule or other suitable delivery means and delivered by an antigen-specific MAb, for effective inactivation of a targeted protein. The compositions can also be applied in consumer products. For instance, the composition can be used as a crucial ingredient in various disinfectants aimed at pathogenic proteins, such as prion.

### A. Proteins and Protein Inactivation

1. Proteins Certain embodiments of the present invention pertain to methods of inactivating a protein or methods of cleaving a disulfide bond of a protein. The term “protein” refers to compounds comprised of one or more chains of amino acids connected by peptide linkages; and includes compounds containing any such fundamental peptide structure, e.g., oligopeptides and polypeptides, as well as oligo- and polypeptides covalently linked to matrices or polymeric supports. The protein may include any number of consecutive amino acid residues.

As used herein, an “amino molecule” refers to any amino acid, amino acid derivative or amino acid mimic as would be known to one of ordinary skill in the art. In certain embodiments, the residues of the protein are sequential, without any non-amino molecule interrupting the sequence of amino molecule residues. In other embodiments, the sequence may comprise one or more non-amino molecule moieties. In particular embodiments, the sequence of residues of the protein may be interrupted by one or more non-amino molecule moieties.

Accordingly, the term “protein” encompasses amino molecule sequences comprising at least one of the 20 common amino acids in naturally synthesized proteins, and includes any modified or unusual amino acid known to those of ordinary skill in the art.

The protein may be a protein made by a natural source, or may be a protein that has been chemically synthesized by any method known to those of ordinary skill in the art.

2. Mechanism of Protein Inactivation

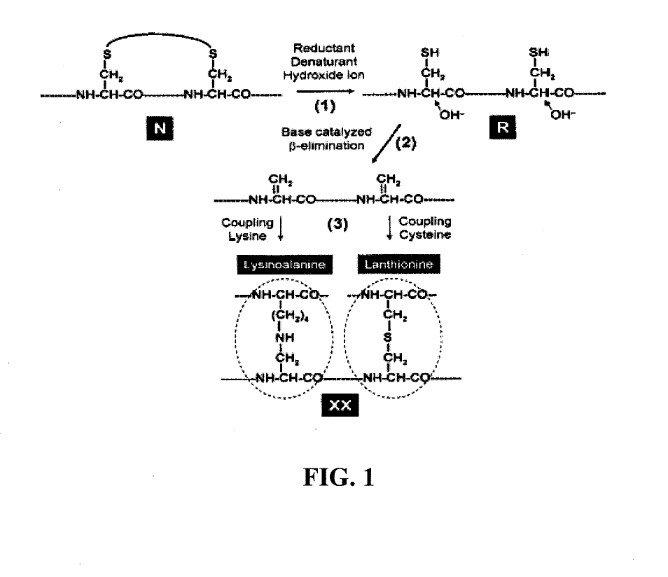

In this present invention, the mechanism of inactivation of disulfide-containing proteins may comprise three stages of consecutive chemical reactions. First, the concerted actions of denaturant, reductant and hydroxide ion, cause a rapid and quantitative reduction of disulfide bonds of the native protein. This leads to the formation of fully reduced (inactive) protein. Second, the presence of hydroxide ion triggers an irreversible destruction of sulflhydryl groups of reduced protein or disulfide bonds of any residual amount partially reduced protein (if exist) via the mechanism of base catalyzed β-elimination (Florence, 1980; Chang, 1991). This leads to the formation of dehydroalanine. Third, the subsequent coupling of dehydroalanine with the side chains of lysine and residual cysteine forms lysinoalanine and lanthionine that inter- and intra-crosslink inactivated protein. The flow chart of these chemical reactions is illustrated in FIG. 1. These three stages of chemical reactions illustrate the mechanism of protein inactivation by the compositions of the present invention. Protein are already inactivated after the first stage of reaction.

### B. Compositions of the Present Invention

The compositions of the present invention may or may not be formulated for pharmaceutical use. For example, as discussed above, the compositions of the present invention may be formulated as disinfectants, such as for disinfecting a non-organic surface such as a floor, a countertop, hospital equipment, or any surface that is included in a area known or suspected to be contaminated with a toxic protein.

In specific embodiments, the compositions of the present invention are formulated in an aqueous solvent. However, solvent systems are contemplated. For example, the solvent may be a hydrophilic solvent, a hydrophobic (organic) solvent, a dipolar aprotic solvent, or a mixture thereof. In some embodiments, the compositions are formulated using a mixture of more than one solvent. For example, the solvent system may include water and one or more water-miscible organic solvents, such as ethanol, acetone, or dimethylsulfoxide (DMSO).

Further, the compositions of the present invention may be formulated with one or more additional ingredients. For example, the additional ingredient may be an additional agent that can be applied in inactivating or cleaving a disulfide bond of a protein. Alternatively, the additional ingredient may be an additional ingredient that has disinfectant capabilities. The additional ingredient may be an ingredient such as a coloring agent, a solubilizing agent, or any additional agent discussed below in relation to pharmaceutical formulations. In some embodiments, the composition includes one or more additional agents that is known or suspected to be involved in inactivation of a protein or cleaving disulfide bonds. For example, the composition may include one or more additional agents such as one or more reductants, one or more denaturants, or one or more additional compounds that generate hydroxide ion. Other additional agents include fragrances or aromas. One of ordinary skill in the art would be able to determine other agents that can be involved in protein inactivation, and any of such agents are contemplated for inclusion in the compositions of the present invention.

### C. Methods of Treatment and/or Prevention of a Disease

“Treatment” and “treating” refer to administration or application of a drug to a subject or performance of a procedure or modality on a subject for the purpose of obtaining a therapeutic benefit of a disease or health-related condition.

The term “therapeutic benefit” used throughout this application refers to anything that promotes or enhances the well-being of the subject with respect to the medical treatment of his condition. This includes, but is not limited to, a reduction in the frequency or severity of the signs or symptoms of a disease. In the context of the present invention, a “therapeutic benefit” may be a reduction in the signs and symptoms associated with exposure to a toxin or a prion.

A “disease” or “health-related condition” can be any pathological condition of a body part, an organ, or a system resulting from any cause, such as infection, genetic defect, and/or environmental stress. The cause may or may not be known. Examples of such conditions include illness due to toxin exposure or any of the prion-related diseases discussed above.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition. Thus, for example, administration of a pharmaceutically effective dose of a composition of the present invention can be given to a subject to block the onset of signs and symptoms of exposure to a toxin or prion following exposure.

The subject can be a subject who is known or suspected of being free of a particular disease or health-related condition at the time the relevant preventive agent is administered. The subject, for example, can be a subject with no known disease or health-related condition (i.e., a healthy subject). In some embodiments, the subject is a subject at risk of developing a particular disease or health-related condition. For example, the subject may be a victim of a bioterrorist attack who has been exposed to a toxin or prion.

### D. Nanocapsules

1. Nanocapsules in General

In certain aspects of the invention, nanocapsules are provided which are targeted to a protein or tissue of interest in a subject. For example, the nanocapsule may be targeted to a toxin, or to a prion protein. Potentially, any type of nanocapsule could be used. In some embodiments, the nanocapsule has a diameter of from 1 nm to 500 nm. In non-limiting embodiments of the invention, the nanocapsule has a diameter of about 50 nm, about 100 nm, about 150 nm, or about 200 nm. It is contemplated that the nanocapsules of the invention may have diameters of about 10 nm, 20 nm, 30 nm, 40 nm, 60 nm, 70 nm, 80 nm, 90 nm, 110 nm, 120 nm, 130 nm, 140 nm, 160 nm, 170 nm, 180 nm, 190 nm, 200 nm, 225 nm, 250 nm, 275 nm, 300 nm, 325 nm, 350 nm, 375 nm, 400 nm, 425 nm, 450 nm, 475 nm, 450 nm, as well as intermediates such as 5 nm, 15 nm, 17 nm, 38 nm, 240 nm and the like.

In some aspects of the invention, the nanocapsule is a pillared construct. In yet other aspects of the invention, the nanocapsule is polymer-based. In still other aspects of the invention, the nanocapsule is a micelle. In some aspects of the invention, the nanocapsule is a nanotubule. In further aspects of the present invention, the nanocapsule is a liposome. The nanocapsules of the invention may be composed of inorganic materials or organic materials. In some specific aspects of the invention, the nanocapsules may be composed of lipids such as phospholipids.

For example, a carbon-based cage structures could be used. Cage-like structures can be formed of, for example, ultra-fine fullerene such as C₆₀ crystallite having diameters in the range of 5 to 50 nm. Bioactive payloads, such as an aliquot of a composition of the present invention that includes a reducing agent, a hydroxide ion, and a denaturant, are enclosed in these structures. Methods for producing the structures are disclosed in U.S. Pat. No. 5,648,056, the entire disclosure of which is specifically incorporated herein by reference. Nanocapsules may also be formed of charged particles of materials including clay and other pillared compounds, which can be linked with short-chain linking molecules to form secondary cage-like structures.

The nanocapsules can range in size from several nanometers to several micrometers in diameter. For example, the nanocapsule may be about 1 nm to about 100 μm in size. In other embodiments, the nanocapsule may be about 1 nm to about 500 nm in size. In further embodiments, the nanocapsule may be about 1 nm to about 100 nm in size.

2. Timed or Triggered Release of Nanocapsule Payloads

In certain embodiments of the invention, the use of nanocapsules designed for sustained, triggered or timed release is contemplated. For example, nanocapsules may be designed to release payloads upon contact with a given signal. In this way, nanocapsule payloads are targeted a site where they are needed to treat or prevent disease or health related conditions associated with exposure to a particular protein.

Such a signal could be endogenous or externally administered. An external signal could be used to cause release of the nanocapsule payloads by, for example, using a chemical signal or physical signal. Examples of physical signals include administration of ultrasound or heat. In this manner the signal could be administered only to the site where treatment with the bioactive factor is needed, maximizing delivery of the factor to the site where needed and minimizing exposure to other parts of the body. Sustained release nanocapsule formulations could also be used. In this manner the efficacy of treatment may be maximized by maintaining therapeutic levels of the bioactive factor over time, without the need for continual administrations of the nanocapsules.

Temporally pulsed release of nanocapsules is also specifically contemplated. This could be achieved, for example, by administration of several types of nanocapsules having different delayed release characteristics. Such temporally pulsed techniques may yield benefits beyond those available with sustained release formulations.

3. Targeted Delivery of Nanocapsules

In some embodiments of the invention, a targeting ligand is surface bound to the nanocapsules. For example, targeted delivery of therapeutics via nanocapsules can occur by passive mechanisms. Passive targeting occurs when nanocapsules extravasate through damaged vasculature to accumulate in tumors and inflamed tissues (Wu et al., 1993). Accumulation increases by improving circulation half-life and by preventing nanocapsules interaction with serum components.

### E. Pharmaceutical Compositions and Formulations

1. Components

In certain aspects of the current invention, pharmaceutical compositions are provided for delivering a pharmaceutically effective amount of a composition of the present invention to a patient or subject in need thereof. Pharmaceutical compositions of the present invention thus comprise an effective amount of a composition of the present invention that includes a reducing agent, a denaturant, and hydroxide ion and any other desired components such as a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate.

In certain embodiments, the composition includes one or more pharmaceutically acceptable carriers. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the compositions of the present invention, its use is contemplated.

2. Method of Administration

Any method known to those of ordinary skill in the art can be applied in delivering a pharmaceutically effective amount of a composition of the present invention to a subject in need. The composition can be delivered non-invasively as a therapeutic or as a countermeasure designed to prevent disease or health-related conditions.

In particular embodiments, the composition is delivered in nanocapsules. The preparation of a pharmaceutical composition that includes a nanocapsule will be well known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

The composition of the present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically (such as via a spray), locally, inhalation (e.g., aerosol inhalation), injection, infusion, continuous infusion, localized perfusion bathing target cells directly, via a catheter, via a lavage, in cremes, in, lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference). In certain aspects of the invention, non-invasive administration techniques in particular may be used advantageously, for example, intranasal administration.

3. “Effective Amount”

An “effective amount” as used herein refers to an amount of a composition of the present invention that includes a reducing agent, a denaturant, and a hydroxide ion that is sufficient to treat or prevent a disease in a subject.

As set forth above, the disease may be one associated with toxic or poisonous proteins, such as a disease associated with toxin exposure or a prion-associated disease. Thus an “effective amount” is one that preferably reduces the amount of symptoms of the condition in the infected patient by at least about 20%, more preferably by at least about 40%, even more preferably by at least about 60%, and still more preferably by at least about 80% relative to untreated subjects. For example, the efficacy of a compound can be evaluated in an animal model system that may be predictive of efficacy in treating the disease in humans, such as the model systems such as those described in the examples or any of those known to one of skill in the art.

The actual dosage amount of a pharmaceutical composition of the present invention administered to a patient can be determined by physical and physiological factors such as body weight, severity of condition, the type of disease or health-related condition being treated or prevented, previous or concurrent therapeutic interventions, idiopathy of the patient and on the route of administration. This amount may also be adjusted based on the disease or health-related condition to be prevented or treated. One advance of the current invention is that targeting allows usage of doses lower than required using non-targeted treatments. In addition, targeting avoids toxicity at sites where delivery of the composition of the present invention is not required.

The practitioner responsible for administration will, in any event, determine the concentration of reducing agent, denaturant, and hydroxide ion in the composition of the present invention that is to be delivered for the individual subject. In certain embodiments, pharmaceutical compositions may comprise, for example, an overall concentration of at least about 0.1% of reducing agent or denaturant, including, for example, about 0.1% to about 75%, 0.1% to about 50%, 0.1% to about 25%, 0.1% to about 10%, 0.1% to about 5%, 0.1% to about 3%, 0.1% to about 1%, 1% to about 10% and about 5% to about 15% of reducing agent or denaturant.

In other non-limiting examples, a dose may also comprise about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, and about 1000 mg/kg/body weight or more of reducing agent or denaturant per administration, and any range derivable therein. In non-limiting examples of a derivable range from the numbers listed herein, a range of about 5 mg/kg/body weight to about 100 mg/kg/body weight of reducing agent or denaturant, about 5 microgram/kg/body weight to about 500 milligram/kg/body weight or reducing agent or denaturant, etc., can be administered in nanocapsule payloads, based on the numbers described above.

In embodiments in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. It will be necessary that such a carrier does not disrupt the nanocapsules prior to delivery to a patient. The proper fluidity of the composition can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In some cases, it will be preferable to include isotonic agents, such as, for example, sugars, sodium chloride or combinations thereof.

In certain embodiments, nanocapsules are prepared for administration by such routes as oral ingestion. In these embodiments, the nanocapsules may comprise, for example, capsules (e.g., hard or soft shelled gelatin capsules) containing a pharmaceutically effective amount of a composition of the present invention.

In certain further embodiments, nanocapsules and other formulations for oral administration may comprise one or more binders, excipients, disintegration agents, lubricants, flavoring agents, and combinations thereof. Such a composition may comprise, for example, one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Sterile injectable solutions of the present pharmaceutical compositions may be prepared, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

Nanocapsules can generally entrap compounds in a stable and/or reproducible way. To avoid side effects due to intracellular polymeric overloading, such ultrafine particles (sized around 0.1 μm) should be designed using polymers able to be degraded in vivo. Biodegradable polyalkyl-cyanoacrylate nanocapsules that meet these requirements are contemplated for use in the present invention, and/or such particles may be easily made.

In some embodiments, administration of a pharmaceutically effective amount of a composition is via a spray or aerosol. Administration to the respiratory tract may be effected for example using a nebulizer or an aerosol inhaler. For the inactivation of proteins in or on the mucosal membranes of living animals, it is intended that aqueous solutions (with or without thickeners) are applied to the membranes in liquid droplet form, or in spray form where it produces a “bathing mist”. Alternatively, liquid compositions may be inhaled as aerosol sprays either via mouth or nose.

In certain embodiments of the present invention, the methods of treatment or prevention further involve identifying a subject in need of treatment or prevention. Any method known to those of ordinary skill in the art can be used to identify a subject in need, including taking a history, performing an examination, and so forth.

### F. Kits

1. Kits for Pharmaceutical Administration to a Subject

Certain embodiments of the present invention pertain to a kit that includes a predetermined amount of a composition of the present invention and a suitable container means. In some embodiments, the kit includes one or more containers or vials containing a predetermined amount of a compositions of the present invention. For example, in some embodiments, the kit includes one or more vials or containers that include nanoparticles as set forth above.

The container means of the kits will generally include at least one vial, test tube, flask, bottle, syringe or other container means, into which a component may be placed, and preferably, suitably aliquoted. Where there are more than one components in the kit, the kit also will generally contain a second, third or other additional container into which the additional components may be separately placed. However, various combinations of components may be comprised in a vial. The kits of the present invention also will typically include a means for containing the composition and any other reagent containers in close confinement for commercial sale. Such containers may include injection or blow-molded plastic containers into which the desired vials are retained.

Kits of the present invention may include pharmaceutical compositions for delivery of bioactive factor-containing nanocapsules targeted to toxic or poisonous proteins. Such kits will generally contain, in suitable container means, a pharmaceutically acceptable formulation of nanocapsules. The kit may have a single container means, and/or it may have distinct container means for each compound.

When the components of the kit are provided in one and/or more liquid solutions, the liquid solution is an aqueous solution, with a sterile aqueous solution being particularly preferred. The compositions may also be formulated into a syringeable composition. In which case, the container means may itself be a syringe, pipette, and/or other such like apparatus, from which the formulation may be injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent, such as a solvent containing hydroxide ion. It is envisioned that the solvent may also be provided in another container means in the kit.

Irrespective of the number and/or type of containers, the kits of the invention may also comprise, and/or be packaged with, an instrument for assisting with the injection/administration and/or placement of the ultimate nanocapsule containing composition within the body of an animal. Such an instrument may be a syringe, pipette, forceps, and/or any such medically approved delivery vehicle.

2. Kits for Spray Application of the Composition of the Present Invention

A pharmaceutically effective amount of a composition comprising a reducing agent, a denaturant, and a hydroxide ion may be comprised in a suitable container means for spray application to a surface or for inhalation by a subject.

The container means of the kits will generally include at least one container means, into which a vial or container for spray application is aliquoted. The kit container may include more than one vial or container means for spray application. The vial or container means may include a device that is suitable for generating a spray from a liquid. Any such device known to those of ordinary skill in the art is contemplated by the present invention. The spray, for example, may be an aerosol.

In some embodiments, a predetermined amount of a composition of the present invention is provided in a single container means in a kit of the present invention. The kit may include more than one container means. The container means may be configured for spray application of the composition, such as to a surface. In other embodiments of the present invention, the kit includes multiple container means into which a predetermined amount of each component of the composition of the present invention is contained. Thus, for example, a kit may include a first container means for a reducing agent, a second container means for a denaturant, and a third container means for a solution containing hydroxide ion, and a fourth container means into which the components of each of the previous container means can be combined. The fourth container means can be configured with a spray nozzle or other applicator device known to those of ordinary skill in the art for spray application of the composition, such as to a surface suspected of contamination by a toxic or poisonous protein.

### EXAMPLES

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### High pH and High Concentration of Hydroxide Ion Accelerate the Reduction of Disulfide Bonds and Trigger Covalent Destruction of Sulfydryl Groups

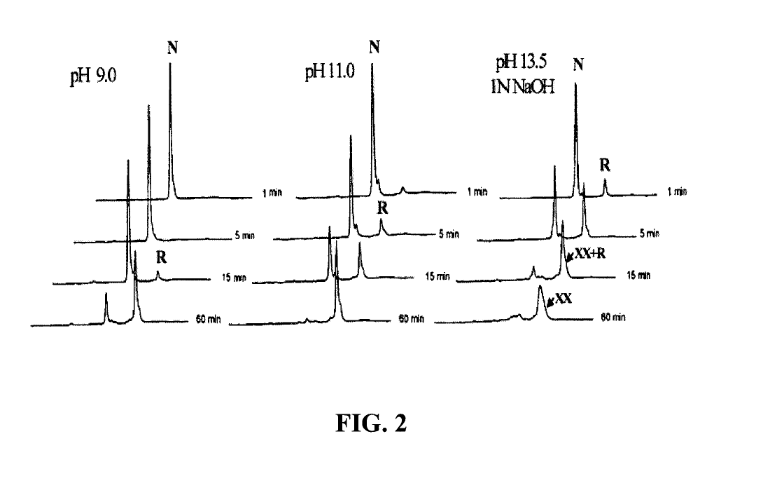

Reduction of protein disulfide bonds by a reducing agent (such as β-mercaptoethanol, dithiothreitol or tris-carboxyethyl-phosphine) is typically performed at pH 8.0-8.5. The increase of pH can significantly accelerate the reduction of disulfide bonds of native proteins. Hirudin (a 3-disulfide protein) is used as an example here. Native hirudin was incubated with 20 mM, 50 mM or 100 mM of DTT at 22° C. in solutions of pH 9.0, 11.0 and 13.5 respectively. The reactions were quenched at different time points with 10 volumes of 4% aqueous trifuoroacetic acid and analyzed by HPLC (FIG. 2). The three disulfide bonds of native hirudin (N) were reduced collectively, leading to the direct formation of fully reduced hirudin (R) without accumulation of partially reduced intermediate FIG. 2. The rate constants of hirudin reduction were shown to be 3.77×10⁻³ min⁻¹ (pH 9.0), 2.35×10⁻² min⁻¹ (pH 11.0), and 1.07×10⁻¹ min⁻¹ (pH 13.5), respectively. Thus, increase of pH from 9.0 to 13.5 accelerates the rate of reduction of hirudin at room temperature by approximately 30-fold. Similar effect of pH was also observed at DTT concentrations of 20 mM and 100 mM.

More importantly, high concentration of hydroxide ion also destroys sulfhydryl group of reduced hirudin via the mechanism of base catalyzed β-elimination, leading to the irreversible inactivation of reduced hirudin. This is evident from the peak shape of time-course trapped samples incubated at pH 13.5 (FIG. 2, right panel). The fully reduced hirudin (R) is eluted as a sharp peak (5 min sample). Reduced hirudin permanently inactivated by hydroxide ion (XX) consists of complex structures (FIG. 1) and is eluted as a broad peak (60 min sample).

### Example 2

### The Inclusion of Denaturant Further Accelerates the Reduction and Destruction of Disulfide Bonds

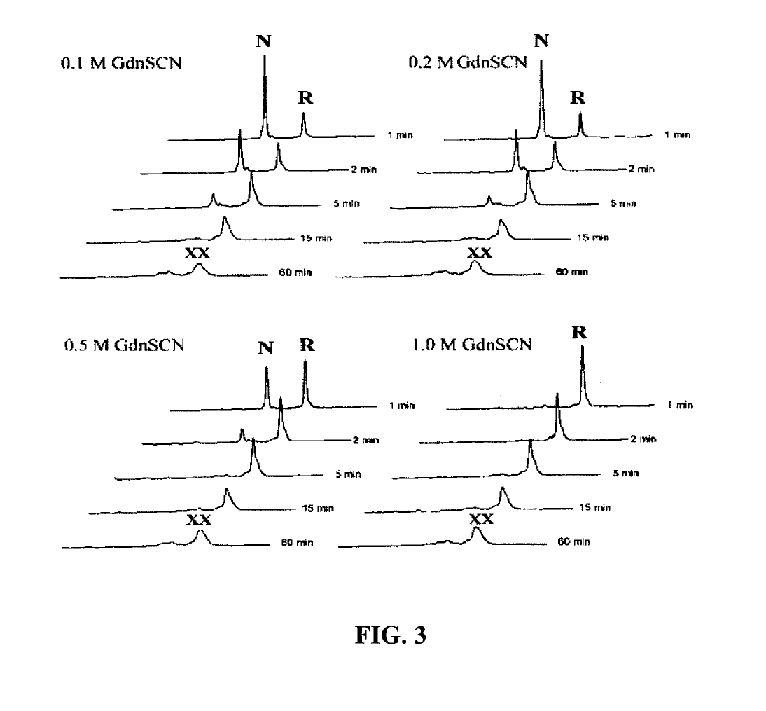

Denaturant is known to destabilize the conformation of native protein and decrease the covalent stability of disulfide bonds against reduction. In addition to the optimized pH (13.5) for hirudin reduction described above, denaturant (GdnCl and GdnSCN) was included to further accelerate the rate of reduction. Native hirudin (N) was incubated at 22° C. in solutions comprising 1N NaOH, 50 mM DTT and increasing concentrations of GdnCl (0.2M-2M) or GdnSCN (0.1M-I M). The reactions were quenched at different time points with 10 volumes of 4% aqueous trifuoroacetic acid and analyzed by HPLC. The results (FIG. 3) show that inclusion of 0.2M and 0.5M of GdnSCN further accelerates the reduction of hirudin by 2.5-fold and 5-fold respectively. In the presence of 1M of GdnSCN, the reduction of hirudin completes within one minute. The potency of GdnCl is approximately 50% of that of GdnSCN.

These data thus demonstrate that in a solution consisting of 1 N NaOH (pH 13.5), 50 mM DTT and 1M GdnSCN, hirudin is fully reduced and inactivated within 1 min at room temperature. The evolution of peak shape further indicates the transformation of R-hirudin to XX-hirudin within about 15 min.

### Example 3

### Analysis of the Reversibility of Inactivated Proteins

Fully reduced and denatured proteins (R) are known to be universally inactivated. However, they are still capable of structural renaturation and restoring their biological activity via oxidative folding. At the stage when free cysteines of R-protein undergo base catalyzed β-elimination, they can be pronounced as irreversibly inactivated. Once cysteines are covalently ravaged (even if only fraction of them), the protein is no longer able to refold and renature. Thus, it is essential to establish a method to measure the reversibility of inactivated protein. This is achieved by: (1) removing the inactivated protein from the denaturant, reductant and hydroxide ion by gel filtration; (2) reconstituting the inactivated protein in a Tris-HCl buffer (0.1M, pH 8.4) containing GSH/GSSG (1 mM/0.5 mM); (3) incubating the inactivated protein at 22° C. overnight, followed by sample analysis using HPLC or activity based assay. If the inactivated protein still contains fraction of R-protein, they will refold and will be recovered as N-protein. If the inactivation is quantitative and irreversible, no N-protein will be recovered.

### Example 4

### Structural Analysis of Irreversibly Inactivated Proteins

Proteins that are irretrievably inactivated by the described method are designated as XX-proteins. These proteins consist of heterogeneous structures of denatured monomers, dimers and trimers etc., that are intra- and inter-crosslinked by lysinoalanine and lanthionine (FIG. 1). Their structural properties can be analyzed by the following methods: (1) SDS-PAGE and MALDI mass spectrometry to determine the molecular mass; (2) Amino acid composition analysis to determine the content of lysinoalanine and lanthionine; (3) Peptide mapping to determine the extent of inter-crosslinking.

### Example 5

### Inactivation of Hirudin by a Composition of the Present Invention

Hirudin is a leech-derived thrombin-specific inhibitor isolated from the leech Hirudo Medicinalis (Markwardt and Walsmann, 1958). It comprises 3 disulfide bonds and 65 amino acids (Dodt et al. 1983). Hirudin is also one of the most stable small proteins. Hirudin is very stable. At 8 M urea and 6 M GdnCl, only 14% and 67% of hirudin exist in a denatured state. This unique stability renders hirudin an excellent candidate for demonstrating the effectiveness of the present invention.

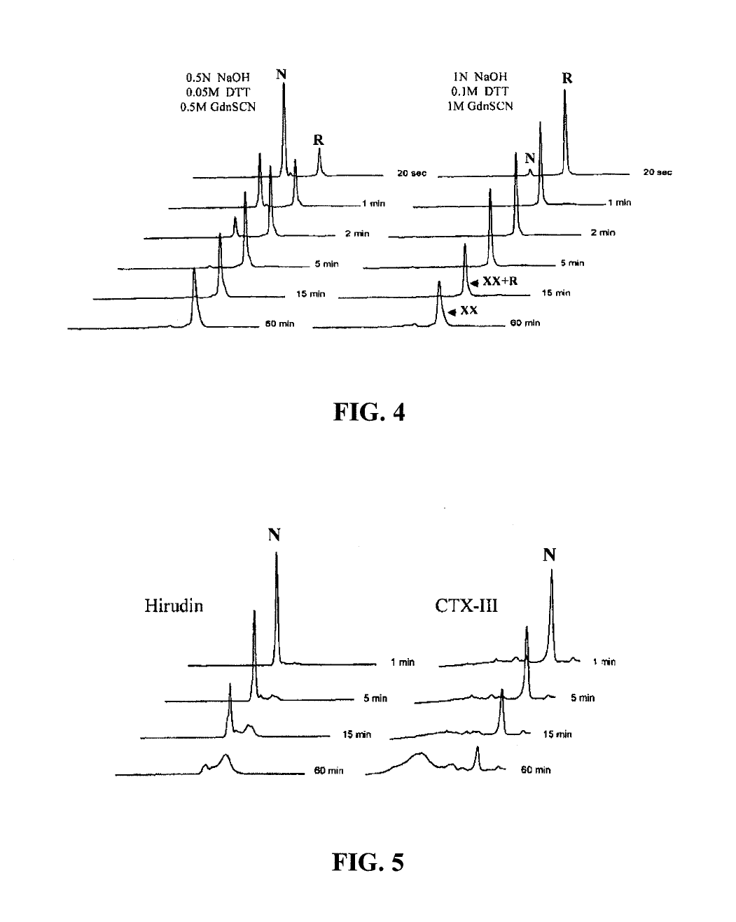

N-Hirudin (1 mg/ml) was treated with a composition that included denaturant (1M GdnSCN), reductant (0.1M DTT) and hydroxide ion (1N NaOH) at 22° C. At different time points, aliquots of treated sample were quenched with 10 volumes of 4% aqueous trifuoroacetic acid and analyzed for their structural and functional properties. For HPLC analysis, the acidified samples were applied directly (FIG. 4). For the analysis of reversibility of inactivated hirudin, the acidified samples were processed as described above in the specification. For anticoagulant activity assay, the inactivated hirudin was purified by gel filtration and analyzed for its anti-amidolytic activity based on its ability to inhibit α-thrombin from digesting chromozym TH (a p-nitroaniline based substrate). For structural characterization, the inactivated hirudin was purified by gel filtration and analyzed for its molecular mass, amino acid composition and peptide mapping (Chymotryptic digestion).

The data obtained from HPLC analysis and anti-amidolytic assay indicate that N-hirudin is almost quantitatively reduced and inactivated within about 20 seconds (FIG. 4). Mass analysis reveals that the permanently inactivated hirudin (XX-Hirudin) consists of heterogeneous monomers (˜50%)˜dimmers (˜30%), trimers (˜15%) and tetramers (˜5%). Amino acid composition analysis recovers 0.6 mole (20%) of cystine, 1.5 moles of lanbthionine and 0.9 mole of lysinoalanine per mole of XX-Hirudin (Chang, 1991). The reversibility test demonstrate that about 50% of the 15 min treated hirudin and >96% of the 60 min treated hirudin are irretrievable (FIG. 5).

### Example 6

### Application of a Composition of the Present Invention in the Inactivation of Cardiotoxin

Cardiotoxin III (CTX-III) is isolated from the highly poisonous Taiwan Cobra (Naja naja afra). It is a basic (pH>10), small molecular weight (6.8 kDa), all β-sheet protein cross-linked by four disulfide bridges (Yu et al., 1994; Kumar et al., 1996). Solution structure of CTX-III revealed that it is a “three finger” shaped protein with three loops emerging from a globular head (Bhaskaran et al., 1996). Mid-point denaturation of CTX-III requires 2.4M of GdnCl (Chang et al., 1998).

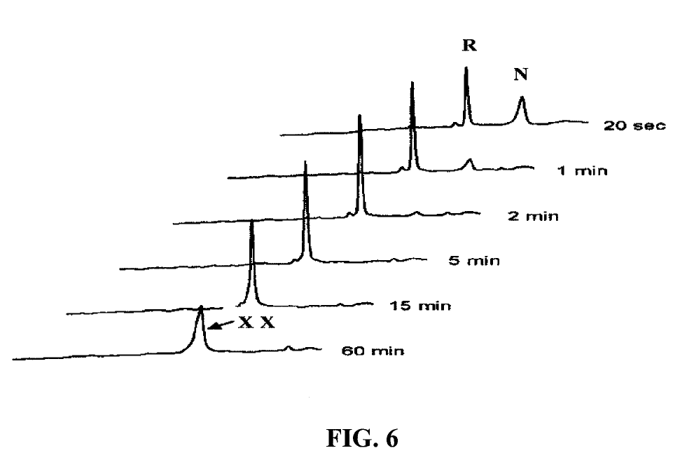

CTX-III (1 mg/ml) was treated with a composition that included denaturant (1M GdnSCN), reductant (0.1M DTT) and hydroxide ion (1N NaOH) at 22° C. At different time points, aliquots of treated sample were quenched with 10 volumes of 4% aqueous trifuoroacetic acid and analyzed by HPLC (FIG. 6). The results show that native CTX-III is fully reduced and inactivated within about 90 seconds. The reversibility test further demonstrates that about 50% of the 15 min treated CTX-III and 85% of the 60 min treated CTX-III are irretrievable (FIG. 5).

### Example 7

### Application of a Composition of the Present Invention in the Inactivation of BPTI, EGF, PCI, SLPI, αIFN and RNase A

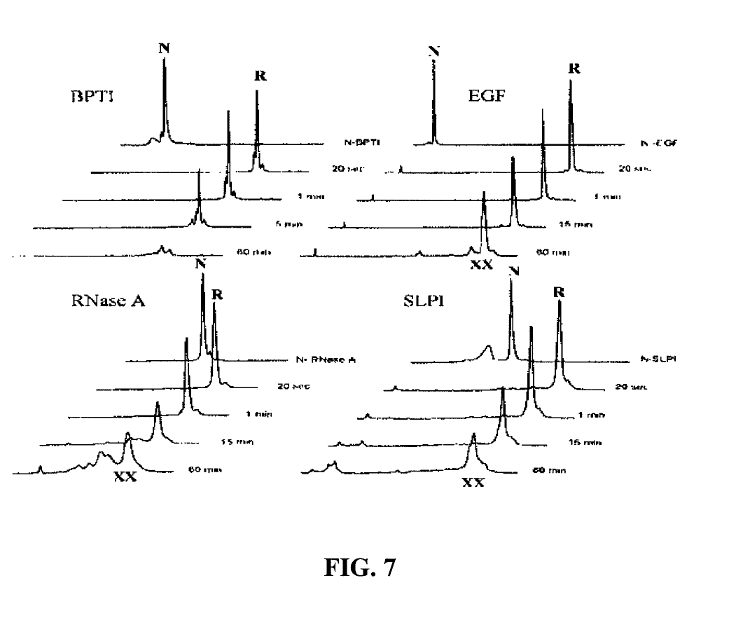

A composition of the present invention was further applied to the inactivation of six other different proteins, including bovine pancreatic trypsin inhibitor (BPTI, 3 disulfides), human epidermal growth factor (EGF, 3 disulfides), bovine ribonuclease A (RNase A, 4 disulfides), leech secretory leucocyte protease inhibitor (SLPI, 8 disulfides), potato Carboxypeptidase inhibitor (PCI, 3 disulfides) and bovine α-interferon (αINF, 2 disulfides). All six native proteins were shown to be quantitatively reduced and inactivated within 20 seconds (FIG. 7) at room temperature. It is noted that BPTI is extremely stable. The mid-point denaturation of BPTI requires a solution containing near saturated GdnCl (7.5M) (Chang and Ballatore, 2000).

### Example 8

### Application of a Composition of the Present Invention in the Depolymerization of Aggregates of Prion Protein

Prion disease inflicts both animals (Mad Cow disease, scrapie of sheep, etc.) and human (Creutzfeldt-Jacob disease) (Gajdusek, 1977; Prusiner, 1999; Chesebro, 1999). The chemical event that underlies the cause of prion disease is the conversion (conformational change) and aggregation of a host derived cellular prion protein (PrP^{C}) to form the infectious aggregates of scrapie prion protein (PrP^{SC}) (Cohen and Prusiner, 1998; Horiuchi and Caughey, 1999). A polymerized form of recombinant mouse prion protein (designated as mPrP-Z) with molecular mass of approximately 340,000 has been generated in vitro at acidic pH (pH 2-5) in the presence of selected concentrations of denaturant (2M GdmCI or 5M urea) (Lu and Chang, 2002). MPrP-Z bears partial structural properties of scrapie prion. It is stable in the acidic solution after removal of denaturant and can be isolated and purified using reversed phase HPLC or size-exclusion HPLC. Isolated mPrP-Z is completely stable in lyophilized form when stored at −20° C. for up to 60 days. This could be verified by analysis of lyophilized samples after reconstitution in the same acidic solution. MPrP-Z also remains stable when incubated at pH 4 (50 mM sodium acetate) in the absence of denaturant for at least 48 hours (Lu and Chang, 2002).

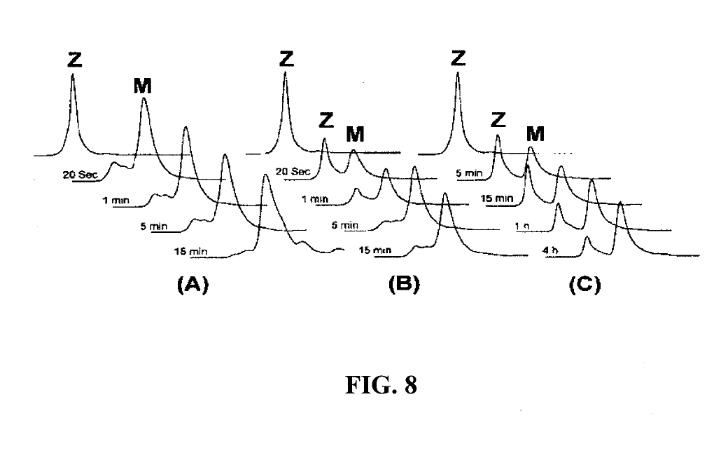

The ability of the invented composite to dissolve the mPrP-Z aggregates was evaluated. Purified mPrP-2 (1 mg/ml) was incubated at 22° C. in three different solutions that comprise: (A) 1N NaOH (pH 13.5), 100 mM DTT and 1M GdnSCN; (B) 0.1M Tris-HCl (pH 8.0), 100 mM DTT and 6M GdnCI; and (C) 0.1M Tris-HCl (pH 8.0) and 6M GdnCI. The reactions were trapped at different time points by sample acidification and analyzed by size-exclusion chromatography (FIG. 8A, B). The results demonstrate that mPrP-Z aggregates can be rapidly (within ˜20 see) dissolved (de-polymerized) by the tested compositions of the present invention (FIG. 8A). At pH 8.0, even coupled with the action of 6M GdnCl, which is about 3-fold more potent than 1M GdnSCN, complete de-polymerization of mPrP-Z occurred after only 4 min of sample incubation (FIG. 8B).

All of the compositions and/or methods disclosed and claimed herein can be made and executed without undue experimentation in light of the present disclosure. While the compositions and methods of this invention have been described in terms of some embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,401,511
U.S. Pat. No. 5,603,872
U.S. Pat. No. 5,889,155
U.S. Pat. No. 5,648,056
U.S. Prov. Appln. 60/351,701
Antibodies: A Laboratory Manual, Cold Spring Harbor Laboratory, Cold Spring Harbor Press, Cold Spring Harbor, N.Y., 1988.
Audi et al., Clinical Rev., 294:2342-2351, 2005.
Bhaskaran et al., J. Mol. Biol., 235:1291-1 301, 1994.
Bigalke and Rummel, Toxicology, 214:210-220, 2005.
Chang, J. Biol. Chem., 266:10839-10843, 1991.
Chang and Ballatore, FEBS Lett., 473:183-1 87, 2000.
Chang et al., Biochemistry, 37:36745-6751, 1998.
Chesebro, Neuron., 24:503-506, 1999.
Cohen and Prusiner, Annu. Rev. Biochem., 67:793-819, 1998.
Creighton, Methods Enzymol., 131:83-106, 1986.
Dodt et al., FEBS Left., 165:180-183, 1983.
Florence, Biochem. J., 189:507-520, 1980.
Forssen and Willis, Adv. Drug Delivery Reviews, 29:249-271, 1998.
Gajdusek, Science, 197:943-960, 1977.
Horiuchi and Caughey, Structure Fold. Des., 7:R231-240,1999.
Kumar et al., Advances in Exptl. Med. & Biol., 391:1 15-1 29, 1996.
Lu and Chang, Biochem. J., 364:81-87, 2002.
Mantis, Adv. Drug Delivery Rev., 57:1424-1439, 2005.
Markwardt and Walsmann, Hoppe-Seylefs Z. Phsiol. Chem., 312:85-98, 1958.
Pace et al., In: Protein Structure, Creighton (Ed.), IRL Press, 31:1-329, 1989.
Prusiner, Proc. Natl. Acad. Sci. USA, 95:13363-13383, 1999.
Remington's Pharmaceutical Sciences, 18th Ed. Mack Printing Company, pp. 1289-1329, 1990.
Spackman et al., J. Biol. Chem., 235:648-659, 1960.
Volkin and Klibanov, In: Protein Function, Creighton (Ed.), IRL Press, 1-24, 1989.
Waxdal et al., Biochemistry, 7:1959-1 966, 1968.
White, Methods Enzymol., 25:387-392, 1982.
Yu et al., J. Toxin Toxicol. Rev., 13:291-315, 1994.

What is claimed is:

1. A method for inactivating a protein, comprising contacting the protein with a composition comprising a reducing agent, a denaturant, and a hydroxide ion, wherein the pH of the composition is about 10.0 to about 14.0 and wherein contacting results in inactivation of the protein.

2. The method of claim 1, wherein the reducing agent is selected from the group consisting of a thiol, a phosphine, and a phosphite.

3. The method of claim 2, wherein the reducing agent is a thiol selected from the group consisting of dithiothreitol (DTT), 5,5′-dithiobis-(2-nitrobenzoic acid), ethanedithiol, 2-mercaptoethanol, 2-mercaptoethylamine, and thioglycolic acid.

4. The method of claim 2, wherein the reducing agent is a phosphine selected from the group consisting of tris-carboxyethylphosphine, trimethyl phosphine, triethyl phosphine, triphenyl phosphine, and tributylphosphine.

5. The method of claim 2, wherein the reducing agent is triethyl phosphite.

6. The method of claim 1, wherein the denaturant is urea, thiourea, guanidinium chloride, imidazole, formamide, dimethylsulfoxide, or a thiocyanate.

7. The method of claim 6, wherein the thiocyanate is guanidine thiocyanate.

8. The method of claim 1, wherein the protein is a prion, and wherein the method is further defined as a method for inactivating a prion.

9. The method of claim 1, wherein the protein is a toxin, and wherein the method is further defined as a method for inactivating a toxin.

10. The method of claim 9, wherein the toxin is ricin, abrin, botulinum toxin, cholera toxin, snake venom toxin, cardiotoxin, diphtheria toxin, Bacillus larval toxin, yeast killer toxin, K1 killer toxin, Cerebratulus toxin, pertussis toxin, hemolysin toxin, microbial-mucosal toxin, Shiga toxin, Helicobacter pylori vacA toxin, anthrax toxin, or tetanus toxin.

11. The method of claim 1, wherein contacting comprises spraying the composition on the protein, applying the composition to the protein with an applicator, or immersing the protein into the composition.

12. The method of claim 1, further defined as a method of treating a subject, wherein the protein is in the subject or on a surface of the subject, comprising administering to the subject a pharmaceutically effective amount of the composition.

13. The method of claim 12, wherein the subject is a human.

14. The method of claim 12, wherein administering comprises topical, aerosol, local, intravenous, intracardiac, intradermal, intralesional, intrathecal, intracranial, intrapericardial, intraumbilical, intraocular, intraarterial, intraperitoneal, intratumor, subcutaneous, intramuscular, or intravitreous administration.

15. The method of claim 12, wherein the composition is topically applied to a surface of the subject.

16. The method of claim 12, wherein the composition is formulated as a nanocapsule.

17. The method of claim 16, wherein the compound is a protein, and wherein the nanocapsule comprises an antibody directed against the protein attached to the surface of the nanocapsule

18. A method for cleaving a disulfide bond of a protein, comprising contacting the protein with a composition comprising a reducing agent, a denaturant, and a hydroxide ion, wherein the pH of the composition is about 10.0 to about 14.0 and wherein contacting results in inactivation of the protein.

19. The method of claim 18, wherein the protein is a toxin.

20. The method of claim 19, wherein the toxin is ricin, abrin, botulinum toxin, cholera toxin, snake venom toxin, cardiotoxin, diphtheria toxin, Bacillus larval toxin, yeast killer toxin, K1 killer toxin, Cerebratulus toxin, pertussis toxin, hemolysin toxin, microbial-mucosal toxin, Shiga toxin, Helicobacter pylori vacA toxin, anthrax toxin, or tetanus toxin.

21. The method of claim 18, wherein the protein is a prion.

22. The method of claim 18, wherein the protein comprises 1-20 disulfide bonds.

23. The method of claim 18, further defined as a method for treating or preventing a prion-related disease in a subject, wherein contacting the prion with the compound results in treatment or prevention of a prion-related disease in the subject.

24. The method of claim 23, wherein the prion-related disease is Creutzfeldt-Jakob Disease, bovine spongiform encephalopathy, variant, Gerstmann-Straussler-Scheinker Syndrome, kuru, scrapie, or fatal familial insomnia.

25. The method of claim 18, wherein the reducing agent is selected from the group consisting of a thiol, a phosphine, and a phosphite.

26. The method of 52, wherein the reducing agent is a thiol selected from the group consisting of dithiothreitol (DTT), 5,5′-dithiobis-(2-nitrobenzoic acid), ethanedithiol, 2-mercaptoethanol, 2-mercaptoethylamine, and thioglycolic acid.

27. The method of claim 25, wherein the reducing agent is a phosphine selected from the group consisting of tris-carboxyethylphosphine, trimethyl phosphine, triethyl phosphine, triphenyl phosphine, and tributylphosphine.

28. The method of claim 25, wherein the reducing agent is a triethyl phosphite.

29. The method of claim 18, wherein the denaturant is urea, thiourea, guanidinium chloride, imidazole, formamide, dimethylsulfoxide, or a thiocyanate.

30. The method of claim 18, wherein the concentration of hydroxide ion is about 0.1N to about 10N.

31. The method of claim 30, wherein the concentration of hydroxide ion is about 0.5N to about 1.5N.

32. The method of claim 18, further defined as a method for disinfecting a surface that has been exposed to an infectious agent, wherein the infectious agent comprises a protein that includes a disulfide bond and wherein the composition is sprayed onto the surface.

33. The method of claim 18, further defined as a method for detoxifying a surface that has been exposed to a toxin, wherein the toxin comprises a disulfide bond and wherein the composition is sprayed onto the surface.

34. The method of claim 33, wherein the surface is organic or non-organic.

35. A composition comprising a reducing agent, a denaturant, and a hydroxide ion, wherein the pH of the composition is about 10.0 to about 14.0.

36. The composition of claim 35, wherein the reducing agent is selected from the group consisting of a thiol, a phosphine, and a phosphite.

37. The composition of claim 36, wherein the reducing agent is a thiol selected from the group consisting of dithiothreitol (DTT), 5,5′-dithiobis-(2-nitrobenzoic acid), ethanedithiol, 2-mercaptoethanol, 2-mercaptoethylamine, and thioglycolic acid.

38. The composition of claim 36, wherein the reducing agent is a phosphine selected from the group consisting of tris-carboxyethylphosphine, trimethyl phosphine, triethyl phosphine, triphenyl phosphine, and tributylphosphine.

39. The composition of 36, wherein the reducing agent is a triethyl phosphite.

40. The composition of claim 35, wherein the denaturant is urea, thiourea, guanidinium chloride, imidazole, formamide, dimethylsulfoxide, or a thiocyanate.

41. The composition of claim 35, wherein the composition is formulated as a liquid, a gel, a foam, a spray, a mist, or a vapor.

42. The composition of claim 35, further comprising a thickener, a corrosion inhibitor, a polymer, a humectant, a surfactant, or an antimicrobial.
